(19) United States
(12) Patent Application Publication
(10) Pub. No.: US 2018/0161121 A1
(43) Pub. Date: Jun. 14, 2018

(54) RADIOPAQUE COMPOSITE WIRE FOR MEDICAL APPLICATIONS AND METHOD OF MAKING A RADIOPAQUE COMPOSITE WIRE

(71) Applicant: Cook Medical Technologies LLC, Bloomuington, IN (US)

(72) Inventors: James Butler, Aherlow, Co. Tipperary (IE); Melissa Lonn, Lafayette, IN (US); Keith R. Milner, West Lafayette, IN (US); John Neilan, Gort, Co. Galway (IE)

(73) Assignee: Cook Medical Technologies LLC, Bloomington, IN (US)

(21) Appl. No.: 15/837,203

(22) Filed: Dec. 11, 2017

Related U.S. Application Data
(60) Provisional application No. 62/433,329, filed on Dec. 13, 2016.

(51) Int. Cl. A61B 90/00; C22F 1/16; B32B 15/01; C22F 1/10; B21C 1/02; A61B 17/22; A61B 17/221; A61B 17/34; A61C 7/20; A61F 2/01; A61F 2/07; A61B 17/00; A61F 2/86; A61M 25/00

(52) U.S. Cl. CPC A61B 90/39; A61M 2025/09141; B32B 15/01; C22F 1/10; B21C 1/02; A61B 17/22031; A61B 17/221; A61B 17/3415; A61C 7/20; A61F 2/013; A61F 2/07; A61B 17/0057; A61F 2/86; A61M 25/005; A61M 25/0012; A61B 2017/00358; A61B 2017/00867; A61B 2090/3966; A61C 2201/005; A61F 2002/016; A61F 2250/0098; C22F 1/16

(57) ABSTRACT

A radiopaque composite wire for medical applications has a core comprising a rare earth metal, an outer layer comprising a nickel-titanium alloy disposed over the core, and a controlled diffusion zone between the core and the outer layer. The controlled diffusion zone includes at least one compound phase comprising (a) the rare earth metal and (b) nickel and/or titanium.

### RELATED APPLICATION

The present patent document claims the benefit of priority under 35 U.S.C. § 119(e) to U.S. Provisional Patent Application Ser. No. 62/433,329, which was filed on Dec. 13, 2016, and is hereby incorporated by reference in its entirety.

### TECHNICAL FIELD

The present disclosure is related generally to biocompatible wire for medical applications and more particularly to a radiopaque composite wire.

### BACKGROUND

Nickel-titanium alloys are commonly used for the manufacture of intraluminal biomedical devices, such as self-expandable stents, stent grafts, embolic protection filters, and stone extraction baskets. Such devices may exploit the superelastic or shape memory behavior of equiatomic or near-equiatomic nickel-titanium alloys, which are commonly referred to as Nitinol.

As a result of the poor radiopacity of nickel-titanium alloys, however, such devices may be difficult to visualize from outside the body using non-invasive imaging techniques, such as x-ray fluoroscopy. Visualization is particularly problematic when the intraluminal device is made of fine wires or thin-walled struts. Consequently, a clinician may not be able to accurately place and/or manipulate a Nitinol stent or basket within a body vessel.

Traditional approaches to improving the radiopacity of nickel-titanium medical devices include the use of radiopaque markers or coatings. For example, gold markers attached to ends of a stent may guide the positioning of the device and delineate its length during an x-ray procedure. Alternatively, a medical device may be plated, clad or otherwise coated with gold or another heavy metal to create a radiopaque surface or outer layer. In another approach, a dense cylinder comprising a metal such as gold or platinum may be included within the lumen of a stent to produce a radiopaque core. These approaches to improving radiopacity may have shortcomings, however. In some cases, markers may be easily dislodged or may undesirably increase the delivery profile of the device, and radiopaque cores comprising noble metals may be expensive to fabricate.

### BRIEF SUMMARY

A radiopaque composite wire for medical applications has a core comprising a rare earth metal, an outer layer comprising a nickel-titanium alloy disposed over the core, and a controlled diffusion zone between the core and the outer layer. The controlled diffusion zone includes at least one compound phase comprising (a) the rare earth metal and (b) nickel and/or titanium.

A method of making a radiopaque composite wire for medical applications includes hot working a composite billet comprising a tube disposed about a rod, where the tube comprises a nickel-titanium alloy and the rod comprises a rare earth metal. The hot working is carried out at a temperature at which controlled diffusion between the nickel-titanium alloy and the rare earth metal occurs so as to form a hot worked composite billet comprising a controlled diffusion zone between an outer layer comprising the nickel-titanium alloy and a core comprising the rare earth metal. The controlled diffusion zone includes at least one compound phase comprising (a) the rare earth metal and (b) nickel and/or titanium. The hot worked composite billet is cold drawn through a die to form a radiopaque composite wire of a predetermined diameter.

According to another embodiment, the method of making the radiopaque composite wire includes cold drawing a composite billet comprising a tube disposed about a rod through a die, where the tube comprises a nickel-titanium alloy and the rod comprises a rare earth metal. A radiopaque composite wire having a core comprising the rare earth metal and an outer layer comprising the nickel-titanium alloy is thereby formed. After the drawing, the radiopaque composite wire is annealed to relieve strain. The annealing is carried out at a temperature at which controlled diffusion between the nickel-titanium alloy and the rare earth metal occurs. Thus, a controlled diffusion zone between the core and the outer layer is formed in the composite wire, where the controlled diffusion zone includes at least one compound phase comprising (a) the rare earth metal and (b) nickel and/or titanium.

### BRIEF DESCRIPTION OF THE DRAWINGS

FIG. 1 provides a schematic of an exemplary radiopaque composite wire including a core comprising a rare earth metal, an outer layer comprising a nickel-titanium alloy, and a controlled diffusion zone between the core and the outer layer.

FIGS. 2A-2C show cross-sectional schematics of an exemplary radiopaque composite wire before and after laser machining to form a stent structure. Due to the presence of the controlled diffusion zone, which acts as a diffusion-bonded interface between the core and the outer layer, delamination is avoided.

### DETAILED DESCRIPTION

A radiopaque composite wire that includes a controlled diffusion zone (or layer) between a nickel-titanium outer layer and a rare earth core is described herein. The composite wire, which is highly radiopaque and may exhibit superelastic behavior or remain martensitic in the human body, can be used for any of a number of implantable or insertable medical devices. The controlled diffusion zone can inhibit unwanted diffusion between the core and the outer layer during processing while allowing diffusion of elements that promote the formation of ductile phases. The controlled diffusion zone may also act as a ductile, bonded interface between the core and the outer layer that can improve the mechanical integrity and properties of the composite wire.

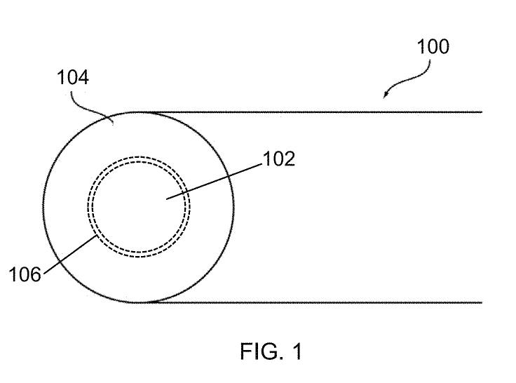

FIG. 1 shows an exemplary radiopaque composite wire 100 for medical applications that includes a core 102 comprising a rare earth metal, an outer layer 104 comprising a nickel-titanium alloy disposed over the core 102, and a controlled diffusion zone 106 between the core 102 and the outer layer 104. The controlled diffusion zone 106 includes at least one compound phase comprising (a) the rare earth metal and (b) nickel and/or titanium. In other words, the compound phase comprises a chemical compound of: the rare earth metal and nickel; the rare earth metal and titanium; or the rare earth metal, nickel and titanium. In some cases, the controlled diffusion zone 106 may include a plurality of compound phases of different compositions where each compound phase comprises the rare earth metal (RE) along with nickel (Ni) and/or titanium (Ti). The controlled diffusion zone 106 may take the form of a uniform or nonuniform layer between the core and the outer layer of the radiopaque composite wire, and may provide a ductile bonded interface between the rare earth metal and the nickel-titanium alloy. Typically, the controlled diffusion zone 106 is continuous about an entire circumference of the core and has a thickness in a range from about 5 angstroms to about 50 microns, or from about 5 angstroms to about 5 microns.

The compound phase(s) of the controlled diffusion zone 106 may be formed during fabrication of the composite wire 100 by a controlled diffusion process that may depend on processing temperature, the chemistry of the rare earth core 102, the processing environment, and/or other factors. As described below, the controlled diffusion process may take place during hot working of a composite billet which is then mechanically drawn down to form the radiopaque composite wire 100. Also or alternatively, controlled diffusion may take place during the annealing that follows cold drawing to form the composite wire.

The compound phase may be, for example, a rare earth-rich compound that includes a greater amount of rare earth metal (e.g., greater than 50 at. % RE) in comparison with the other element(s) of the compound (e.g., nickel, titanium, or nickel and titanium). Generally speaking, the rare earth-rich compound may have a composition REₓNi_{y}, REₓTi_{y}, REₓNi_{y}Ti_{z} or REₓ(Ni_{1-w},Ti_{w})_{y} where x is greater than y or y+z, and where w is between 0 and 1. In the case of stoichiometric compounds, x, y and z have integer values, and in the case of non-stoichiometric compounds, x, y and z may have non-integer values. In one example, when the core comprises Er as the rare earth metal, the compound phase may comprise an erbium-rich compound including Er and Ni, such as Er₃Ni, Er₃Ni₂, or Er₃(Ni₁₋ₓ,Tiₓ), where 0<x<1.

In another example, the compound phase may be a titanium-rich compound that includes a greater amount of titanium (e.g., greater than 50 at. % Ti) in comparison with the other element(s) of the compound (e.g., the rare earth metal, or the rare earth metal and nickel). For example, the titanium-rich compound may have a composition TiₓRE_{y} or TiₓRE_{y}Ni_{z}, where x is greater than y or y+z. In the case of stoichiometric compounds, x, y and z have integer values, as indicated above, and in the case of non-stoichiometric compounds, x, y and z may have non-integer values. In yet another example, the compound phase may be a nickel-rich compound that includes a greater amount of nickel (e.g., greater than 50 at. % Ni) in comparison with the other constituent(s) of the compound (e.g., the rare earth metal, or the rare earth metal and titanium). In this case, the nickel-rich compound may have a composition NiₓRE_{y} or NiₓRE_{y}Ti_{z}, where x is greater than y or y+z.

The controlled diffusion zone 106 may further include an elemental rare earth (RE) phase comprising the rare earth metal. For example, when the core comprises erbium, the controlled diffusion zone 106 may further include an elemental erbium phase comprising Er, which may be beneficial since an excess of Er can improve the ductility of the controlled diffusion zone, minimizing the likelihood of cracking or delamination during processing or use of the composite wire. The elemental RE phase may have the crystallographic structure of the pure rare earth metal but may in some cases include one or more elements (e.g., diffused Ni or Ti in solid solution) in addition to the rare earth metal. Alternatively, the elemental RE phase may consist essentially of the rare earth metal and no additional elements, other than incidental impurities.

The rare earth metal of the core 102 may be selected from among the following elements, which are found in the lanthanide and actinide series of the periodic table: La, Ce, Pr, Nd, Pm, Sm, Eu, Gd, Tb, Dy, Ho, Er, Tm, Yb, Lu, Ac, Th, Pa and/or U. Sc and Y, which are sometimes considered rare earth elements, may also be employed as the rare earth metal. Preferred rare earth metals for the core 102 include Er and Lu. The core 102 may comprise more than one rare earth metal and/or other element(s) that can form an alloy or compound with the rare earth metal. Thus, the core 102 may comprise a metal, alloy or compound that includes at least one rare earth element selected from Sc, Y, the lanthanide series elements, and the actinide series elements, as set forth above.

To help control diffusion during hot working (and/or annealing) as the controlled diffusion zone 106 is formed, it may be advantageous for the core 102 to include, in addition to the rare earth metal, a small amount of nickel and/or titanium. For example, the core 102 comprising the rare earth metal may further include up to about 10 wt. % Ni and/or up to about 10 wt. % Ti.

In another example, the core 102 may include, in addition to the rare earth metal, an element selected from among Ag, Cu, Au, Ir and Rh that forms an alloy or compound with the rare earth metal. Such rare earth alloys or compounds may exhibit better mechanical properties than the rare earth metal alone (e.g., increased strength and hardness without a detrimental loss of ductility). A class of rare earth-based intermetallic compounds that may have a good balance of strength and ductility include, for example, yttrium-silver (YAg), yttrium-copper (YCu), dysprosium-copper (DyCu), cerium-silver (CeAg), erbium-silver (ErAg), erbium-gold (ErAu), erbium-copper (ErCu), erbium-iridium (ErIr), holmium-copper (HoCu), neodymium-silver (NdAg), yttrium-iridium (YIr) and yttrium-rhodium (YRh). Alternatively, the core 102 may include only the rare earth metal and any incidental impurities.

Advantageously, the rare earth metal of the core 102 is highly radiopaque while also being compatible with magnetic resonance imaging (MRI). A radiopaque material preferentially absorbs incident x-rays and tends to show high radiation contrast and good visibility in x-ray images. MRI compatible materials can be viewed in vivo in MRI scans and images without significant artifacts. The radiopacity of several rare earth elements has been shown to be comparable to that of platinum—a highly radiopaque metal—over the photon energy range from about 40 keV to about 80 keV, as shown by previously obtained absorption coefficient data (as shown in U.S. Pat. No. 9,103,006, entitled “Nickel-Titanium Alloy Including a Rare Earth Element,” which is hereby incorporated by reference in its entirety). Lutetium, which has a higher density than the rare earth elements examined in the '006 patent, is expected to have an even higher radiopacity.

The nickel-titanium alloy of the outer layer 104 may exhibit superelastic and/or shape memory behavior. That is, the nickel-titanium alloy may undergo a phase transformation that allows it to “remember” and return to a previous shape or configuration. More specifically, the nickel-titanium alloy may transform between a lower temperature phase (martensite) and a higher temperature phase (austenite). Austenite is characteristically the stronger phase, and martensite may be deformed up to a recoverable strain of about 8%. Strain introduced in the alloy in the martensitic phase to achieve a shape change may be substantially recovered upon completion of a reverse phase transformation to austenite, allowing the alloy to return to a previous shape. The strain recovery may be driven by the application and removal of stress (superelastic effect) and/or by a change in temperature (shape memory effect). Such alloys are commonly referred to as Nitinol or Nitinol alloys, and they are typically near-equiatomic in composition. Due to the presence of the Ni—Ti alloy outer layer 104, the radiopaque composite wire 100 may behave superelastically and/or utilize the shape memory effect. Alternatively, the nickel-titanium alloy of the outer layer 104 may remain in the martensitic phase during use of the radiopaque composite wire 100. Radiopaque composite wires as described herein may find use in any of a number of medical devices. For example, the radiopaque composite wire may be employed individually or in combination as part of an insertable or implantable medical device, such as, for example, a wire guide, a stent, a stent graft, a torqueable catheter, an introducer sheath, an orthodontic arch wire, a radiopaque marker or marker band, or a manipulation, retrieval, or occlusive device such as a grasper, a snare, a basket (e.g., stone extraction or manipulation basket), a vascular plug, or an embolic protection filter.

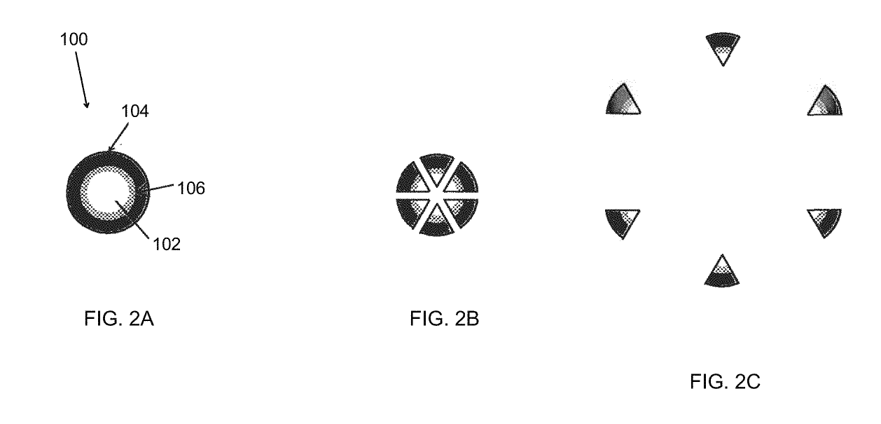

The controlled diffusion zone may function as a ductile, diffusion-bonded interface between the nickel-titanium alloy of the outer layer and the rare earth metal of the core. Thus, the radiopaque composite wire may exhibit excellent structural integrity, workability and mechanical properties in use. For example, as shown schematically in FIGS. 2A-2C, the radiopaque composite wire 100 may undergo laser machining through the cross-section of the wire (FIG. 2B) without delamination in order to form a stent structure (FIG. 2C).

Nickel-rich compositions of the nickel-titanium alloy may be advantageous to ensure that the composite wire 100 exhibits superelastic behavior at body temperature. For use in medical applications in the human body in which superelasticity is desired, it is beneficial for the nickel-titanium alloy of the composite wire to have an austenite start temperature Aₛ below body temperature (e.g., 37° C.) and an austenite finish temperature A_{f} at or below body temperature. Alternatively, if the nickel-titanium alloy is to remain martensitic during use in the body, the nickel-titanium alloy may have an austenite finish temperature A_{f} above body temperature, and the austenite start temperature Aₛ may also be above body temperature. As known to those of skill in the art, austenite start temperature (Aₛ) is the temperature at which a phase transformation to austenite begins upon heating for a nickel-titanium alloy exhibiting an austenitic phase transformation, and austenite finish temperature (A_{f}) is the temperature at which the phase transformation to austenite concludes upon heating. Martensite start temperature (Mₛ) is the temperature at which a phase transformation to martensite begins upon cooling for a nickel-titanium alloy exhibiting a martensitic phase transformation, and martensite finish temperature (M_{f}) is the temperature at which the phase transformation to martensite concludes upon cooling.

For example, the nickel-titanium alloy may have from greater than 50 at. % Ni to about 52 at. % Ni, or from about 50.6 at. % Ni to about 50.8 at. % Ni. Titanium and any incidental impurities may account for the balance of the nickel-titanium alloy. In some cases, the nickel-titanium alloy may also include a small amount of an additional alloying element (AAE) (e.g., from about 0.1 at. % AAE to about 10 at. % AAE) to enhance the superelastic or other properties of the nickel-titanium alloy. The additional alloying element may be selected from among B, Al, Cr, Mn, Fe, Co, Cu, Zn, Ga, Ge, Zr, Nb, Mo, Tc, Ru, Rh, Pd, Ag, Cd, In, Sn, Sb, Hf, Ta, W, Re, Os, Ir, Pt, Au, Hg, Tl, Pb, Bi, Po, V, and Mischmetal.

A suitable volume ratio of the rare earth core 102 to the Ni—Ti outer layer 104 may be determined based on the desired radiopacity of the composite wire 100. Commercially available drawn filled tubing (DFT) wire including a nickel-titanium shell and a platinum core may be used as a benchmark, since platinum is highly radiopaque and such wires are employed for medical device applications. In some commercial products, the platinum core may account for about 10 vol. % or about 30 vol. % of the DFT wire. As discussed above, the radiopacity of several rare earth elements has been shown to be comparable to or higher than that of platinum over the photon energy range from about 40 keV to about 80 keV. Assuming a similar or better radiopacity, it is possible to determine a suitable amount of rare earth material to incorporate into the core 102. One approach is to use the same volume fraction of rare material that has proven effective with platinum (e.g., from 10 vol. % to about 30 vol. %). In another approach, assuming that a comparable amount of the rare earth material in atomic percentage provides the same or better radiopacity than platinum, one can calculate a desired volume percentage of the rare earth metal.

For example, a commercial wire including a platinum core (30% by volume) and a Ni-rich (51 at. % Ni-49 at. % Ti) nickel-titanium shell includes about 28.70 at. % Pt, along with 36.65 at. % Ni and 34.65 at. % Ti. Assuming that a comparable atomic percentage of the rare earth material provides the same or better radiopacity than Pt, the desired volume percentage of the rare earth metal in a radiopaque composite wire may be calculated. An assumption is made that the thickness of the controlled diffusion zone is much smaller than the diameter of the core and the thickness of the outer layer, and thus is not included in these calculations. The core is understood to have a cylindrical shape with the outer layer radially surrounding the core along the length thereof. The results are summarized in the tables below.

In the case of a radiopaque composite wire 100 with a core 102 comprising Er or Lu and an outer layer 104 comprising a Ni—Ti alloy (51 at. % Ni), a suitable volume percentage of the rare earth metal may range from about 18 vol. % to about 46 vol. %, although percentages outside this range may also be used. Generally speaking, to achieve a suitable radiopacity from the radiopaque composite wire described herein, the rare earth core 102 may account for from about 15 vol. % to about 60 vol. % of the composite wire 100.

TABLE 1
Biomedical grade binary Nitinol

| | | |
|---|---|---|
| Atom Type | At % To Wt % | Vol % |

| | | | |
|---|---|---|---|
| Ni | 51.00 | 56.05 | 39.21 |
| Ti | 49.00 | 43.95 | 60.79 |

TABLE 2
30% Pt core DFT compared to Er and Lu of similar radiopacity

| | | |
|---|---|---|
| Atom Type | Wt. % To At. % | Vol. % |

| | | | |
|---|---|---|---|
| Ni | 22.86 | 36.65 | 27.71 |
| Ti | 17.64 | 34.65 | 42.29 |
| Pt | 59.50 | 28.70 | 30.00 |
| Ni | 24.99 | 36.65 | 21.19 |
| Ti | 19.28 | 34.65 | 32.33 |
| Er | 55.74 | 28.70 | 46.48 |
| Ni | 24.36 | 36.65 | 21.57 |
| Ti | 18.79 | 34.65 | 32.91 |
| Lu | 56.85 | 28.70 | 45.53 |

TABLE 3
10% Pt core DFT compared to Er and Lu of similar radiopacity

| | | |
|---|---|---|
| Atom Type | Wt. % To At. % | Vol. % |

| | | | |
|---|---|---|---|
| Ni | 40.64 | 46.27 | 35.37 |
| Ti | 31.73 | 44.27 | 54.62 |
| Pt | 27.63 | 9.46 | 10.00 |
| Ni | 42.31 | 46.27 | 32.08 |
| Ti | 33.04 | 44.27 | 49.54 |
| Er | 24.65 | 9.46 | 18.38 |
| Ni | 41.84 | 46.27 | 32.31 |
| Ti | 32.66 | 44.27 | 49.88 |
| Lu | 25.50 | 9.46 | 17.81 |

To fabricate the radiopaque composite wire described above, a composite billet that includes a tube comprising a nickel-titanium alloy disposed about a rod comprising a rare earth metal may be formed. The composite billet may be hot worked at a temperature at which controlled diffusion between the nickel-titanium alloy and the rare earth metal occurs, and an extruded composite billet comprising a controlled diffusion zone between a core comprising the rare earth metal and an outer layer comprising the nickel-titanium alloy is formed. As described above, the controlled diffusion zone includes at least one compound phase comprising (a) the rare earth metal and (b) nickel and/or titanium. Preferably, the controlled diffusion zone is continuous about an entire circumference of the core. The hot worked composite billet may be cold drawn through a die, typically under ambient conditions, to reduce the diameter of the billet and obtain a radiopaque composite wire having any of the characteristics described above.

The hot working of the composite billet may entail hot extrusion. In one example, the assembly may undergo direct extrusion without a protective sleeve or indirect extrusion canned in a protective sleeve (e.g., a copper alloy sleeve) at an elevated temperature. To avoid formation of undesirable oxide phases during hot working, it may be advantageous to hot work the composite billet under vacuum conditions. For example, the protective sleeve or can may be pumped out to a suitable vacuum level and sealed prior to extrusion.

Suitable temperatures for hot working are below the melting temperatures of the nickel-titanium alloy (about 1300° C.) and the rare earth metal, which may be from about 800° C. to about 1500° C., depending on the element. Thus, a hot working (e.g., hot extrusion) temperature in the range from about 500° C. to about 1000° C. may be suitable. For example, hot extrusion may be carried out at a temperature (e.g., furnace temperature) in the range from 600° C. to 800° C. Experiments have shown that adiabatic heating during extrusion (e.g., due to friction) may increase the temperature experienced by the composite billet beyond the temperature of the furnace. Thus, even with a furnace temperature below the minimum temperature known to induce diffusion and compound phase formation between the rare earth metal and the nickel (or titanium), a controlled diffusion zone may be formed during hot working. The minimum temperature to induce diffusion and form a compound phase may be determined from the appropriate phase diagram. In the case of erbium, for example, the minimum temperature for phase formation with nickel is 765° C., as can be observed from the nickel-erbium phase diagram. Due to adiabatic heating, suitable temperatures for hot working radiopaque composite billets comprising an Er core may thus be below 765° C. (e.g., 750° C. and below, or 725° C. and below). Generally speaking, suitable temperatures (e.g., furnace temperatures) for hot working radiopaque composite billets as described herein may be below the minimum temperature at which diffusion and compound phase formation are known to occur, as determined by the appropriate phase diagram. Hot working may be carried out in a time duration of several minutes or less. Typical extrusion ratios (calculated by dividing the starting cross-sectional area by the extruded cross-sectional area) may range from at least about 2:1 up to about 27:1, or more typically from 2:1 to about 12:1.

The drawing down of the composite billet to form the composite wire may be followed by annealing to relieve strain, and multiple cold drawing passes with dies of decreasing diameters may be used to progressively reduce the diameter of the composite billet to the desired final wire diameter. Typically, from two to ten cold drawing passes and several interpass annealing treatments are carried out. The number of cold drawing passes may depend on the starting billet size and the targeted final diameter of the composite wire. A polycrystalline diamond die may be employed for cold drawing with a molybdenum disulphide or other suitable lubricant to reduce the drawing stress. Generally, at least about 7.5% cold work is imparted to the composite billet during each drawing pass. As would be recognized by one of ordinary skill in the art, percent (%) cold work provides a measurement of the amount of plastic deformation imparted during mechanical working, where the amount is calculated as a percent reduction in a given dimension. In wire drawing, the percent cold work may correspond to the percent reduction in the cross-sectional area of the billet or wire resulting from a drawing pass. The amount of cold work or area reduction imparted to the composite billet to form the composite wire may reach or exceed about 50%. Typically, the area reduction with each drawing pass is from about 7.5% to about 15%. For example, a 3 mm diameter becomes 2.77 mm in diameter if a 15% area reduction is achieved. The final cold drawing steps may be the most important, and may be carried out with or without interpass anneals. For example, the final cold drawing passes may involve up to a 50% area reduction to obtain the required dimensions and properties, where multiple smaller area reductions are obtained from successive cold drawing passes without interpass annealing.

Annealing may be carried out at a temperature in the range from about 600° C. to about 800° C. The time duration for annealing is typically at least one minute or at least five minutes, and may be as high as 60 minutes or up to 20 minutes. An annealing treatment may soften the strain-hardened composite billet via recrystallization and grain growth. After cold drawing of the composite wire to the desired final diameter, the controlled diffusion zone may be from about 5 angstroms to about 50 microns in thickness, and is more typically from about 5 angstroms to about 5 microns in thickness. Prior to cold drawing, the controlled diffusion zone may have a considerably larger thickness, e.g., from one to three orders of magnitude larger in the composite billet. With each drawing pass, the composite wire is reduced in diameter, and the thickness of the controlled diffusion zone decreases also. As indicated above, it is preferred that the diffusion barrier maintains a continuous interface between the outer layer and the core during hot and/or cold working.

For example, a maximum ratio of the thickness of the controlled diffusion zone to the outer diameter of the Ni—Ti alloy tube may be about 1:10 if significant hot working and cold working are required to reduce the composite billet down in size to fine wire. Typically this ratio is closer to 1:33, but it may be a minimum of 1:50. Large composite billets may be reduced by any of the standard hot working methods, e.g. gyratory forging, extrusion, etc. For production scale processing, the outer diameter of the composite billet is typically about 100 mm in diameter, and thus the controlled diffusion zone may be about 3 mm in thickness (1:33) prior to cold drawing of the composite billet.

It is also contemplated that for some medical device applications or for low volume production, a hot working step may not be required to form the radiopaque composite wire. In such a case, the elevated temperature anneal(s) that follow cold drawing may be carried out under conditions (e.g., temperature, environment, time, as described herein) sufficient to promote diffusion and form a controlled diffusion zone in the composite wire.

To fabricate the composite billet prior to hot working and/or cold drawing, a longitudinal hole may be drilled through an ingot comprising the nickel-titanium alloy to form a tube, and a rod comprising the rare earth metal may be assembled within the tube. The ingot and/or the rod may be formed by melting and casting or powder metallurgy (e.g., spark plasma sintering) methods known in the art, such as those described in U.S. Pat. Nos. 9,074,274, 9,103,006 and 9,212,409, which are hereby incorporated by reference.

Due to the high temperature exposure during hot working and annealing, it is advantageous for the composite billet to comprise materials having similar coefficients of thermal expansion. For example, Nitinol is known to have a coefficient of thermal expansion (CTE) of 11×10⁻⁶/° C., while Er has a CTE of 12.2×10⁻⁶/° C. and Lu has a CTE of 9.9×10⁻⁶/° C. Otherwise, the core may expand rapidly relative to the outer layer (or vice versa) during processing, possibly inducing rupture of the outer layer or fracture of the core.

After cold drawing to form the composite wire, a shape-setting heat treatment may be carried out to impart a “memory” of the desired final shape to the nickel-titanium alloy. The heat treatment may also serve to optimize the properties of the nickel-titanium alloy and alter the phase transformation temperatures. Typically, heat setting temperatures from about 350° C. to about 550° C. are appropriate to set the final shape and optimize the shape memory/superelastic and mechanical properties of the nickel-titanium alloy. Preferably, the heat setting involves annealing the composite wire while constrained in a final shape at a temperature in the range of from about 350° C. to about 550° C. In some cases, heat setting temperatures in the range of from 450° C. to 550° C. may be appropriate.

### Radiopacity

The radiopacity of a material is related to its linear absorption coefficient, μ, which depends on its effective atomic number (Z_{eff}) and density (ρ), and on the energy (E) of the incoming x-ray photons:

$$\frac{μ}{ρ} = const \cdot \frac{{Z}_{eff}^{3}}{{E}^{3}}$$

The linear absorption coefficient μ is proportional to the density ρ of the material, and thus the quantity

$$\frac{μ}{ρ}$$

is a material constant known as the mass absorption coefficient and expressed in units of cm² gm⁻¹.

Linear absorption coefficients μ calculated for several rare earth elements and also for platinum for comparison were presented in U.S. Pat. No. 9,103,006, as mentioned above. The figures and data from the '006 patent indicate that the absorption of the rare earth elements tends to peak in the photon energy range of about 40 to 80 keV, with some rare earth elements exceeding the absorption of platinum in this region.

### Magnetic Susceptibility

MRI compatible materials generally have a low magnetic susceptibility. The magnetic susceptibility X of a material can be represented by the ratio of the magnetization M of the material to the applied magnetic field H and is dimensionless:

$$X = \frac{M}{H} \qquad Eq . 1$$

Different materials respond differently to applied magnetic fields H, and thus may have widely varying values of magnetic susceptibility X. As Equation 1 indicates, materials that respond strongly to a magnetic field have a high magnetic susceptibility. Materials are classified as diamagnetic, paramagnetic, or ferromagnetic depending on their response to an applied magnetic field. For example, a susceptibility spectrum may extend from X=−1.0 for diamagnetic superconductors to X>100,000 for soft ferromagnetic materials. In the case of medical devices made of materials having a high magnetic susceptibility (such as stainless steel, which is ferromagnetic), a disturbance in the magnetic field is created around the device when the device is visualized with MRI. As a result, the device may not properly transfer MRI signal information and portions of the magnetic resonance image or scan in the vicinity of the component may become distorted, exhibiting dark or black areas in the image. Accordingly, materials having a high magnetic susceptibility are generally not believed to be MRI-compatible.

A material having a magnetic susceptibility X falling in the range from about −2×10⁻⁵ to about 2×10⁻⁵ may be understood to be MRI compatible. A molar susceptibility Xₘ is defined as equal to X·MW/ρ (cm³/mol), where MW is molecular weight and ρ is density. Values of Xₘ for the rare earth elements are provided in Table 4 below. As can be seen, terbium, dysprosium, holmium, erbium and thulium have relatively high values of molar susceptibility, while rare earth elements such as scandium, yttrium, lanthanum, lutetium, and ytterbium show more promise for applications in which MRI compatibility is important.

TABLE 4
Values of Molar Susceptibility for the Rare Earth Elements

| Rare Earth Element | Xₘ/10⁻⁶ cm³/mol |
|---|---|
| Sc | +295.2 |
| Y | +187.7 |
| La | +95.9 |
| Ce | +2,500 |
| Pr | +5,530 |
| Nd | +5,930 |
| Sm | +1,278 |
| Eu | +30,900 |
| Gd | +185,000 |
| Tb | +170,000 |
| Dy | +98,000 |
| Ho | +72,900 |
| Er | +48,000 |
| Tm | +24,700 |
| Yb | +67 |
| Lu | +182.9 |
| Th | +97 |
| Pa | +277 |
| U | +409 |

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A radiopaque composite wire for medical applications, the radiopaque composite wire comprising:
a core comprising a rare earth metal;
an outer layer comprising a nickel-titanium alloy disposed over the core; and
a controlled diffusion zone between the core and the outer layer, the controlled diffusion zone including at least one compound phase comprising (a) the rare earth metal and (b) nickel and/or titanium.

2. The radiopaque composite wire of claim 1, wherein the controlled diffusion zone is continuous about an entire circumference of the core.

3. The radiopaque composite wire of claim 1, wherein the controlled diffusion zone comprises a thickness in a range from about 5 angstroms to about 50 microns.

4. The radiopaque composite wire of claim 1, wherein the at least one compound phase comprises a rare earth-rich phase.

5. The radiopaque composite wire of claim 4, wherein the rare earth-rich phase has a composition REₓNi_{y}, REₓTi_{y}, REₓNi_{y}Ti_{z} or REₓ(Nii_{1-w},Ti_{w})_{y} where x is greater than y or y+z, and where w is between 0 and 1.

6. The radiopaque composite wire of claim 1, wherein the controlled diffusion zone further comprises an elemental rare earth phase comprising the rare earth element.

7. The radiopaque composite wire of claim 1, wherein the at least one compound phase comprises a titanium-rich phase.

8. The radiopaque composite wire of claim 1, wherein the rare earth metal comprises Er and the compound phase is selected from the group consisting of: Er₃Ni, Er₃Ni₂, and Er₃(Ni₁₋ₓ,Tiₓ), where 0<x<1.

9. The radiopaque composite wire of claim 1, wherein the core further comprises Ni, and wherein the Ni is present in the core at a concentration of about 10 wt. % or less.

10. The radiopaque composite wire of claim 1, wherein the core further comprises Ti, and wherein the Ti is present in the core at a concentration of about 10 wt. % or less.

11. The radiopaque composite wire of claim 1, wherein the core further comprises an additional element selected from the group consisting of: Ag, Cu, Au, Ir and Rh.

12. The radiopaque composite wire of claim 1, wherein the nickel-titanium alloy of the outer layer exhibits superelastic behavior during use in the human body, or
wherein the nickel-titanium alloy of the outer layer remains martensitic during use in the human body.

13. A medical device comprising the radiopaque composite wire of claim 1.

14. The medical device of claim 13 being selected from the group consisting of: wire guide, stent, stent graft, torqueable catheter, introducer sheath, orthodontic arch wire, radiopaque marker or marker band, grasper, snare, basket, vascular plug, and embolic protection filter.

15. A method of making a radiopaque composite wire for medical applications, the method comprising:
hot working a composite billet comprising a tube disposed about a rod, the tube comprising a nickel-titanium alloy and the rod comprising a rare earth metal, the hot working being carried out at a temperature at which controlled diffusion between the nickel-titanium alloy and the rare earth metal occurs, thereby forming a hot worked composite billet comprising a controlled diffusion zone between a core comprising the rare earth metal and an outer layer comprising the nickel-titanium alloy, the controlled diffusion zone including at least one compound phase comprising (a) the rare earth metal and (b) nickel and/or titanium; and
cold drawing the hot worked composite billet through a die to form a radiopaque composite wire of a predetermined diameter.

16. The method of claim 15, wherein the cold drawing of the hot worked composite billet is followed by annealing to relieve strain, and
further comprising multiple passes of the cold drawing and the annealing to form the radiopaque composite wire.

17. The method of claim 15, wherein the temperature of the hot working is below a minimum temperature known for inducing diffusion and compound phase formation between (a) the rare earth metal and (b) the nickel and/or the titanium.

18. A method of making a radiopaque composite wire for medical applications, the method comprising:
cold drawing a composite billet comprising a tube disposed about a rod through a die, the tube comprising a nickel-titanium alloy and the rod comprising a rare earth metal, thereby forming a radiopaque composite wire having a core comprising the rare earth metal and an outer layer comprising the nickel-titanium alloy, and
after the drawing, annealing the radiopaque composite wire to relieve strain, the annealing being carried out at a temperature at which controlled diffusion between the nickel-titanium alloy and the rare earth metal occurs, thereby forming a controlled diffusion zone between the core and the outer layer, the controlled diffusion zone including at least one compound phase comprising (a) the rare earth metal and (b) nickel and/or titanium.

19. The method of claim 18 further comprising multiple passes of the cold drawing and the annealing to form the radiopaque composite wire.

20. The method of claim 18, wherein the temperature of the annealing is below a minimum temperature known for inducing diffusion and compound phase formation between (a) the rare earth metal and (b) the nickel and/or the titanium.
